(19) United States
(12) Patent Application Publication
(10) Pub. No.: US 2008/0299564 A1
(43) Pub. Date: Dec. 4, 2008

(54) Control of Preservation By Biomarkers

(75) Inventor: Bart Jan Frederik Keijser, Hilversum (NL)

Correspondence Address: LUCAS & MERCANTI, LLP, 475 PARK AVENUE SOUTH, 15TH FLOOR, NEW YORK, NY, 10016, US

(73) Assignee: Nederlandse Organisatievoor toegepast natuurwetenschappelijk Onderzoek TNO, Delft (NL)

(21) Appl. No.: 12/089,149

(22) PCT Filed: Aug. 4, 2006
(86) PCT No.: PCT/NL2006/000412

§ 371 Date: Jun. 16, 2008

(30) Foreign Application Priority Data
Oct. 4, 2005 (EP) 05077246.6

(51) Int. Cl. C12Q 1/68; C07H 21/02

(52) U.S. Cl. 435/6; 536/23.1

(57) ABSTRACT

The invention lies in the field of microbiology, more particularly in the field of (food) preservation and testing of viability of microbiological spores. It is shown that measurement of the integrity of both rRNA and mRNA in spores is an accurate indicator of viability. Nucleotide assays then form a significant improvement over the state of the art assays for viability.

The invention lies in the field of microbiology, more particularly in the field of (food) preservation and testing of viability of microbiological spores.

Since the development of pasteurisation no real break-through has been achieved in the field of food preservation. The most common preservation process is heat treatment and the effectiveness of preservation is nowadays determined by a trial and error method. Thus, present preservation methods are directed to a ‘worst case’ scenario, in which the most infected sample is taken as standard for subsequent preservation treatments. One of the reasons for this is that a detailed insight in the process of preservation lacks: current methodologies are not capable of a reliable and quick assessment of the micro-organisms and their viability in relation to the preservation process. Hitherto, the inactivation of contaminating micro-organisms is measured indirectly: by determining the growth of surviving micro-organisms after heat treatment. This method has two large disadvantages:

it is very time consuming (often results are only available after more than two days);

if no survivors are measured, it cannot be determined whether the applied treatment was just barely sufficient or meant an overkill.

The worst case scenario in practice means that often more energy is used for the heat treatment than would have been necessary, because either the contamination was less than expected or the micro-organisms in the sample were more heat-sensitive than those on which the treatment was based. It will be understood that any surplus heat treatment of the product will affect its quality.

Thus, there is a large need for rapid microbiological testing of (heat) treated samples to assess the quality and effect of the preservation treatment.

In the last couple of years genomic techniques have enabled a relatively rapid and specific approach to microbiological assaying. With these techniques it is possible to detect even small amounts of (micro)organisms within a sample, and it has become possible to discriminate between micro-organisms on a species or subspecies level. However, especially in heat (or other stress) treated food, the most important safety challenge is formed by the spores. Spores are formed by spore-forming micro-organisms under growth limiting (stress) conditions. Spore-forming micro-organisms, such as Bacillus and Clostridium, determine for a major part the shelf-life of heat-preservation treated food. One spore is formed from one vegetative bacterium. Each spore is composed of a protoplast, a protoplast membrane, cortex and multiple coat layers (Turnbill, 1996). This multilayered outer shell excludes macromolecules. An additional heat and chemical resistant component in the cortex, peptidoglycan, increases the spore's resiliency (Turnbill, 1996). Bacillus spores central protoplast contains dipicolinic acid, (DPA), the component necessary for high temperature tolerance (Turnbill, 1996). Slieman et al. (2001) found that approximately 10 percent of a spore's dry weight is DPA. DPA exists as a calcium complex. Rosen et al. (1997) developed a terbium chloride assay to identify and quantify endospore concentrations utilizing this DPA-Ca++ complex. The reaction between the calcium dipicolinic acid complex and terbium chloride results in a terbium (III) anion. This terbium anion is photoluminescent when in the presence of the DPA-Ca++ complex and is easily recognized. As mentioned above, the protoplast is enveloped by the cortex, followed by three protein coats (Turnbill, 1996). However, members of the B. cereus group, which includes, B. anthracis, have an additional protective layer, the outer-most exosporium. These multilayered outer structures, which can make up half of the spore's weight, provide protection for the spores from chemical, physical and enzymatic degradation (Turnbill, 1996).

Several species of Bacillus and Clostridium are important to human and animal health. B. anthracis, the agent of anthrax, is a zoonotic disease which primarily affects grazing animals and can also be a dangerous pathogen to humans. B. anthracis spores are known to survive along livestock trails in the United States causing frequent outbreaks in states from Texas to South Dakota (CDC, 2001). Members of the B. cereus group can cause food poisoning in humans and related forms (eg. B. thuringiensis) are used in biological control of insect pests. C. perfringens and C. botulinum are commonly known food-contaminating bacteria. C. perfringens is a Gram-positive square-ended anaerobic (microaerophilic) bacillus classified in Group III of the Family Bacillaceae. This non-motile member of the clostridia forms oval, central spores rarely seen in culture unless grown in specially formulated media, although the spores are produced readily in the intestine. Capsules may be seen in smears from tissue. Sugar reactions (acid and gas) may be irregular. Nitrate is reduced and lecithinase (alpha-toxin activity) can be demonstrated in egg yolk medium (Nagler reaction). Food poisoning from C. perfringens gives rise to abdominal pain, nausea and acute diarrhea 8-24 h after the ingestion of large numbers of the organism, a proportion of which survive the acid conditions of the stomach (Sutton & Hobbs, 1971). The illness is usually brief and full recovery within 24-48 h is normal. However, death occasionally occurs in the elderly or otherwise debilitated patients, e.g. in hospitals or institutions (Smith, 1998). The symptoms of the disease are caused by an enterotoxin. C. perfringens is grouped into 5 types A-E according to the exotoxins (soluble antigens) produced. Types A, C and D are pathogens for humans, types B, C, D and E, and possibly A also, affect animals. The enterotoxin produced by types A and C is distinct from the exotoxins and is responsible for the acute diarrhea that is the predominant symptom of C. perfringens food poisoning. The beta-toxin of type C appears to be the necrotic factor in the disease enteritis necroticans jejunitis (“pig-bel”). Type A strains are responsible for gas gangrene (myonecrosis), necrotizing colitis, peripheral pyrexia, septicaemia as well as food poisoning.

Clostridium botulinum neurotoxin (BoNT) has the capacity to cause disease in essentially all vertebrates. Symptoms may appear in a few hours or take several days to appear. Initial symptoms such as weakness, fatigue and vertigo, are usually followed by blurred vision and progressive difficulty in speaking and swallowing. In type E botulism nausea and vomiting often occur early in the illness and probably contribute to its lower mortality than types A and B. Disturbed vision and difficulty in speaking and swallowing are due to neurological implications involving extra-ocular and pharynx muscles. Weakening of diaphragm and respiratory muscles also occurs and death is usually due to respiratory failure. Specifically neurotransmission of the peripheral nerve system is blocked. The mortality rate has fallen due to early diagnosis, prompt administration of antitoxin, and artificial maintenance of respiration. The illness is serious and full recovery usually takes many months.

C. tetanus causes one of the major diseases still present in developing countries, tetanus. Tetanus has been known to take up to ten years to manifest, but normally, incubation period is a few days to a few weeks. The first signs of the disease include mild muscle contractions at the site of infection as the infection gradually spreads along nerve fibers to the spinal cord and brain stem. Trismus (lockjaw) ensues with continued rigidity and spasms of the extremities. Death occurs when spasms interfere with respiration.

There are several reliable assays used to characterize and identify spores. Genetic identification relies on PCR-DNA sequencing to identify the species by their nucleotide sequences (Hansen, B., Leser, T., Hendriksen, N. 2001. Polymerase chain reaction assay for the detection of Bacillus cereus group cells. FEMS Microbiology Letters 202: 209-213; Kolbert, C., Persing, D. 1999. Ribosomal DNA sequencing as a tool for identification of bacterial pathogens. Microbiology 2:299-305; Lindstrom, M. et al., 2001. Multiplex PCR assay for detection and identification of Clostridium botulinum types A, B, E, and F in food and fecal material. Appl. Environm. Microbiol. 67:5694-5699; US 2003/0203362). Phenotypic identification relies on physiological profiling such as the Sherlock Microbial Identification System (MIDI Inc., Newark, Del.), and BIOLOG, (Biolog, Inc., Hayward, Calif.). The MIDI system utilizes fatty acid analysis of the bacterium, and assigns a numerical value to the results, called a similarity index. This similarity index is then compared to an internal library which chooses the most similar identities for the microbes in question. BIOLOG is a cell-based test for utilization of carbon sources. It requires a 96-well plate with multiple carbon sources from which color changes are compared to a Biolog library to determine the identification of the bacterium. Steady-state fluorescence is a powerful tool for distinguishing differences in molecules and macromolecules and the technique may prove useful in examining spores. Fluorescence spectroscopy utilizes emission peaks to characterize spores. It is a sensitive technique because excitation is performed at a single wavelength. The resulting emission data are recorded at a longer wavelength. Bronk and Reinisch (1993) concluded that initial microbial identification could be generated using fluorescence spectroscopy. All spore assays are influenced by environmental contaminants (Kuske, et al., 1998, Balser, et al. 2002, Gamo et al. 1999) and each would benefit from isolation, concentration and purification.

One technique that may aid in spore isolation and concentration from environmental samples is to use partitioning into hexadecane or some similar hydrophobic material whereby hydrophobic spores would partition into the hydrocarbon and hydrophilic spores would remain in the aqueous phase.

Thus spores are commonly more heat-resistant than the cells of the micro-organism, also because they do not require an active metabolism during the spore stage. Yet, on return of favourable conditions the spores can develop into colonizing micro-organisms and then are able to cause enormous health-risks. Until now no genomics-based techniques have been developed to assay the amount and viability of spores after preservation treatment without the requirement of pregermination of the surviving spores.

### SUMMARY OF THE INVENTION

The invention now provides for an easy and reliable assay to measure the viability and/or detonation (e.g. by damaging) of spores during or after preservation treatment. The assay uses biomarkers, comprising RNAs, more especially ribosomal RNAs (rRNAs) and messenger RNAs (mRNAs). It has now been found that preservation treatments such as a heat treatment or pressure degrades these RNAs and also that the sensitivity towards degradation varies within these ribonucleotides

Thus, the invention provides a method to measure viability of bacterial spores, which have been subjected to a preservation treatment, such as heat, pressure, radiation or combinations of these, chemicals like hypochlorite, benzoates, nitrites and sulphites, and pEF (pulsed electric fields, comprising measuring the degree of degradation of RNA by said treatment in said spores, particularly wherein the RNA is either ribosomal RNA (rRNA), messenger RNA (mRNA) or both, more particularly wherein the treatment is heat treatment.

Another embodiment of the invention is a method to assay the effect of heat preservation methods by performing a method as described above.

Alternatively, the invention provides a method to assay bacterial contamination after heat or radiation preservation by performing a method according to the invention.

The invention also provides a heat preservation method comprising

a) maintaining a sample at a certain temperature for a certain time;
b) performing a method according to the invention.

Another embodiment of the present invention is formed by the use of RNA in microbial spores as a biomarker for viability.

### LEGENDS TO THE FIGURES

FIG. 1 shows survival of B. subtilis 168 spores after heat treatment at the indicated temperatures during the time period depicted on the X-axis. The Y-axis indicates logarithmically the number of colony forming units (cfu).

FIG. 2 shows a Bioanalyzer pseudogel of RNA isolated from B. subtilis 168 spores treated at three different temperatures (90° C., 98° C. and 105° C.) each during four different time periods (2, 5, 10 and 20 minutes), indicating the integrity of the RNA. The left lane represents molecular weight control.

FIG. 3 shows the results of survival counts of two B. subtilis strains (168 and A163) which have been heat treated at the indicated temperatures (X-axis) during a period of 5 minutes. Y-axis as in FIG. 1.

FIG. 4 shows a Bioanalyzer pseudogel of RNA isolated from the spores. Treatments identical as in FIG. 3.

FIG. 5 Shows a heat map of spore mRNAs (in duplo, indicated on the right) measured during germination. Red color indicates presence of transcript, green color indicates absence.

FIG. 6 shows ethidium bromide stained gels of three PCR products (Bs-2 (=coxA), Bs-4 (=ykzE) and Bs-7 (=sspE) obtained through RT-PCR from the spore mRNA. In each panel the left lane shows the molecular weight marker.

FIG. 7 indicates survival of B. subtilis 168 spores after heat treatment at 98° C. for the indicated time periods (X-axis). Y-axis as in FIG. 1.

FIG. 8 depicts the results of the quantitative analysis of degradation of spore rRNAs and mRNAs. The bars indicate the ¹⁰log value of the decrease in RNA concentration measured in treated spores in comparison with untreated spores. Spores and treatments in FIG. 7.

FIG. 9 shows a Bioanalyzer pseudogel of RNA isolated from C. botulinum spores treated at three different temperatures (80° C., 105° C. and 110° C.), the last two during three different time periods (5, 10 and 15 minutes), indicating the integrity of the RNA. The left lane (M) represents molecular weight control. Lane 1 is untreated, Lane 2 is treatment at 80° C. for 10 minutes. Lanes 3, 4 and 5 is treatment at 105° C. for 5, 10 and 15 minutes respectively. Lanes 6, 7 and 8: ibid for 110° C.

FIG. 10 Top panel: Ethidium bromide stained gels showing denaturation products of RNA of B. licheniformis ATCC 14580 after heat treatment at 90° C. for 0, 2, 5, 10 and 20 minutes. Bottom panel: viability counts.

FIG. 11 Top panel: Effect of duration of ultra high pressure treatment on viability of B. subtilis spores. Bottom panel: Bioanalyzer pseudogel. Lanes represent the following treatments:

M: molecular size marker
1: RNA from control sample (untreated spores)
2: RNA from spores subjected to high pressure (600 MPa) for 2 minutes
3: RNA from spores subjected to high pressure (600 MPa) for 10 minutes
4: RNA from spores subjected to high pressure (600 MPa) for 30 minutes

### DETAILED DESCRIPTION OF THE INVENTION

The presence of messenger RNA is microbial spores has been disputed. Some old literature indicates that spores contain mRNAs (Aronson A. J., 1965, Mol. Biol. 13:92-104; Jeng Y. H. and Doi R. H., 1974, J. Bacteriol. 119:514-521), while other authors report an absence (Halvorson H O, Vary J C, Steinberg W. J, (1966). Developmental changes during the formation and breaking of the dormant state in bacteria. Annu Rev Microbiol 20:169-88; R. H. Doi and R. T. Igarashi (1964) RIBONUCLEIC ACIDS OF BACILLUS SUBTILIS SPORES AND SPORULATING CELLS. Bacteriol. February; 87(2): 323-328). However, nowhere in the art identification of spores on basis of mRNA or other RNA sequences is taught.

It has now appeared that Bacillus subtilis spores contain ribosomal RNAs (more particularly the 16S and 23S rRNAs) and also mRNAs for about 20 gene products. The latter finding is surprising, since it was generally thought that only de novo transcription takes place in spores (during germination). This small group of mRNAs is generated during the last phase of sporulation and is in general rapidly degraded during germination. The function of these transcripts and why they are maintained in the spore is as of yet unclear. A list of the mRNAs that are found in spores is given in Table 1.

TABLE 1
List of spore mRNA found in spores of B. subtilis 168.

| Gene Details | Gene name |
|---|---|
| BG14189 | yozQ |
| BG14179 | sspN |
| BG14174 | sspJ |
| BG13937 | ytzC |
| BG13861 | ythQ |
| BG13859 | ythC |
| BG13782 | coxA |
| BG13430 | ymfJ |
| BG13334 | ykzF |
| BG13333 | ykzE |
| BG13008 | yhdB |
| BG12879 | yfhD |
| BG11921 | sspP |
| BG11920 | sspO |
| BG11806 | tlp |
| BG11670 | yqfX |
| BG11600 | yhcV |
| BG11595 | yhcQ |
| BG11592 | yhcN |
| BG10789 | sspE |
| BG10108 | sspF |

The presence of both rRNA and mRNA enables using molecular biological techniques to study survival and/or heat resistance in spores during radiation and heat treatment (e.g. for preservation). One of the findings of this invention is that both rRNA and mRNA are being degraded during heat treatment, which degradation coincides with the loss of viability of the spores. Degradation predominantly depends on the type of micro-organism: in spores of a more heat resistant micro-organism the degradation of the RNA starts at higher temperatures. Thus, the degradation of RNA is an excellent parameter for determination of the viability and/or (sublethal/postmortal) damage of the spores.

Molecular biological assays for the detection of RNA and/or determination of the length of the RNA fragments can now be used to assess the viability of spores in (food and other) samples. This is not only useful during development of preservation methods on basis of heat treatment, but these assays can also be applied in the regular testing of the condition of food samples, e.g. by the commodity inspection department.

Typically, for the molecular biological methods of the invention, first the RNA of the spores has to be isolated from the sample. To enrich the sample for spores, methods which are well known to a person skilled in the art can be used (e.g. Vaerewijck, M. J. M. et al. (2001) J. Appl. Microbiol. 91:1074-1084). Then, spores are lysed and RNA is extracted using commonly known techniques. RNA isolation from spores can, for instance, be done through a commercially available kit, such as the BIO101-FASTRNA Pro Blue kit (QBIOGENE cat no 6025-050). To remove superfluous DNA from the RNA, the extracted sample can be treated further with a DNAse1, such as present in the TURBO DNA-free kit (AMBION, cat no 1907).

Determination of the length of the RNAs can be performed on normal gels, such as denaturing agarose gels, but it is also possible to analyse the integrity of the RNA on a bioanalyzer RNA chip, such as a RNA 6000 Nano LabChip (Agilent Technologies cat no 5065-4476) using a 2100 bioanalyzer (Agilent Technologies, cat no G2940CA). The integrity of the isolated RNA samples is normally derived from the relative intensity of the bands for the ribosomal subunits (16S and 23S). When the RNA is intact the ratio between 23S and 16S is about 2:1. On RNA degradation this ratio shifts usually in favour of 16S RNA and increasingly RNA debris is found.

In order to make detection more sensitive the nucleotides in the sample can be amplified, e.g. through an RNA amplification kit, such as Nucleic Acid Sequence Based Amplification (NASBA). Alternatively, it is also possible to convert the RNA into DNA by using a reverse transcriptase and then to amplify the DNA by PCR techniques. Methods of the invention can in principle be performed by using any nucleic acid amplification method, such as the Polymerase Chain Reaction (PCR; Mullis 1987, U.S. Pat. No. 4,683,195, 4,683,202, en 4,800,159) or by using amplification reactions such as Ligase Chain Reaction (LCR; Barany 1991, Proc. Natl. Acad. Sci. USA 88:189-193; EP Appl. No. 320,308), Self-Sustained Sequence Replication (3SR; Guatelli et al., 1990, Proc. Natl. Acad. Sci. USA 87:1874-1878), Strand Displacement Amplification (SDA; U.S. Pat. Nos. 5,270,184, en 5,455,166), Transcriptional Amplification System (TAS; Kwoh et al., Proc. Natl. Acad. Sci. USA 86:1173-1177), Q-Beta Replicase (Lizardi et al., 1988, Bio/Technology 6:1197), Rolling Circle Amplification (RCA; U.S. Pat. No. 5,871,921), Cleavage Fragment Length Polymorphism (U.S. Pat. No. 5,719,028), Isothermal and Chimeric Primer-initiated Amplification of Nucleic Acid (ICAN), Ramification-extension Amplification Method (RAM; U.S. Pat. Nos. 5,719,028 and 5,942,391), LATE-PCR (Sanchez, J. A. et al (2004) PNAS USA 101:1933-1938) or other suitable methods for amplification of DNA.

For quantitative assays a Real Time-PCR or a semi-RT-PCR can be used. All of these amplification techniques are known by the person skilled in the art, and application of those can be found in the Examples, hereunder.

The detection of the amplification products can in principle be accomplished by any suitable method known in the art. The detection fragments may be directly stained or labelled with radioactive labels, antibodies, luminescent dyes, fluorescent dyes, or enzyme reagents. Direct DNA stains include for example intercalating dyes such as acridine orange, ethidium bromide, ethidium monoazide or Hoechst dyes. Alternatively, the DNA fragments may be detected by incorporation of labelled dNTP bases into the synthesized DNA fragments. Detection labels which may be associated with nucleotide bases include e.g. fluorescein, cyanine dye or BrdUrd.

Amplification can be used to detect the integrity of the RNA sequences because amplification primers can be chosen in such a way that only (nearly) complete, i.e. not yet degraded sequences will be amplified. A suitable way to accomplish this is by choosing a forward amplification primer which corresponds with a sequence at the 5′ end of the sequence which needs to be amplified. Consequently, the backward or reverse primer should be chosen to correspond with a sequence at the 3′ end of the sequence to be amplified. Only if forward and reverse primer anneal to one and the same (intact) RNA stretch amplification will occur; if the target sequence has been degraded by heat or radiation treatment a fragment will at the utmost only contain the recognition site for one of the set of primers and no amplification will take place. Further, a quantitative determination of the amplicons at the 3′ end and the 5′ end gives an indication of the amount of fragmentation of the target nucleic acid.

The term “primer” as used herein refers to an oligonucleotide which is capable of annealing to the amplification target allowing a DNA polymerase to attach thereby serving as a point of initiation of DNA synthesis when placed under conditions in which synthesis of primer extension product which is complementary to a nucleic acid strand is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxy ribonucleotide. The primer must be sufficiently long to prime the synthesis of extension products in the presence of the agent for polymerization. The exact lengths of the primers will depend on many factors, including temperature and source of primer. A “pair of bi-directional primers” as used herein refers to one forward and one reverse primer as commonly used in the art of DNA amplification such as in PCR amplification.

If the nucleotide sequences from the sample are subjected to amplification for the detection of RNA integrity, it is a prerequisite that from each micro-organism (i.e. from each spore) at least one sequence is amplified. Therefore, primer sets should be chosen which recognise all possible micro-organisms present. This can be achieved by identifying conserved sequences, which are shared by all the micro-organisms. Since it is sometimes impossible to cover all micro-organisms with one and the same primer set, it is envisaged that more than one pair of bidirectional primers is needed to be present in de amplification reaction mixture to enable amplification of at least one sequence from all possibly present micro-organisms. Luckily, the 16S and 23S rRNA are rich in stretches of conserved sequences. Alternatively, or in addition to that, it is also possible to use degenerated primers, i.e. primers which do not or less suffer from any mismatches between the primer sequence and the target sequence on the gene to be amplified, thereby allowing for hybridisation of the primer to a target sequence with a lower homology.

Another method to determine the viability and/or amount of damage to the spores is to assess for the ratio of intact DNA versus intact RNA. As already mentioned in the introduction, the spores also contain DNA, which has other temperature sensitivity than RNA (see e.g. Setlow, P. 1995. Mechanisms for the prevention of damage to DNA in spores of Bacillus species. Annu. Rev. Microbiol. 49:29-54; and Setlow, P. 1999. Bacterial spore resistance, p. 217-230. In G. Storz, and R. Hengge-Aronis (ed.), Bacterial stress responses. American Society for Microbiology, Washington, D.C). In a control experiment it can be determined for a particular bacterial strain how the ratio of DNA:RNA develops when the spores are subjected to heat treatments, and at which ratio the spores are lethally damaged or lost their viability. A simple detection of the amounts of DNA and the amounts of RNA can then be used to determine viability and/or damage.

Use of the methods of the invention can of course be found in the food-industry. Not only detection of contamination of actual foodstuffs can be performed through the methods of the invention, these methods also are a useful tool in the development of (new) preservation methods for different types of samples (predominantly food). The methods enable monitoring of preservation processes and quick feedback on the results thereof. Also, as a result of better preservation techniques, next to a decrease of product loss also savings of energy and water can be obtained.

Besides the food industry, the present invention can also be applied in other areas where preservation treatments, such as heat and/or radiation and/or pressure, chemicals, such as hypochlorite, benzoates, nitrites and sulphites, and pEF (pulsed electric field) are used for sterilisation (i.e. killing of micro-organisms). An example of such an area is in the medical environment where surgical instruments, hospital consumables, such as bandages and surgical gloves, and also patient waste material require sterilisation.

### EXAMPLES

### Example 1

### Heat Inactivation of Bacillus subtilis Spores

Spores of Bacillus subtilis 168 [Bacillus genetic stock center: http://www.bgsc.org/BGSCID:] were generated by culture in synthetic MOPS medium (1.32 mM K₂HPO₄, 0.4 mM MgCl₂, 0.276 mM K₂SO₄, 0.01 mM FeSO₄, 1.36 mM CaCl₂, 80 mM MOPS (morpholinepropane sulfonic acid), 4 mM tricine, 3 nM (NH₄)₆Mo₇O₂₄, 0.4 μm H₃BO₃, 30 nM CoCl₂, 10 nM CuSO₄, 10 nM ZnCl₂, 0.1 mM MnCl₂, 20 mM glucose and 10 mM ammoniumchloride). The thus obtained spores were purified with 10 steps of washing with demineralised water at 4° C. The purity of the spores was monitored with a phasecontrast microscope (>99% phase bright). Heat treatment of the spores was done according to Kooiman et al. (“The screw cap tube technique: A new and accurate technique for the determination of the wet heat resistance of bacterial spores”. In: Spore Research, ed: Barker, A. N., Gould G. W. and Wolf, J., 1973, Academic Press, London, pp. 87-92). Survival was counted by plating serial dilutions of the samples in a peptone saline solution on TSA (tryptric soy agar, DIFCO, the Netherlands).

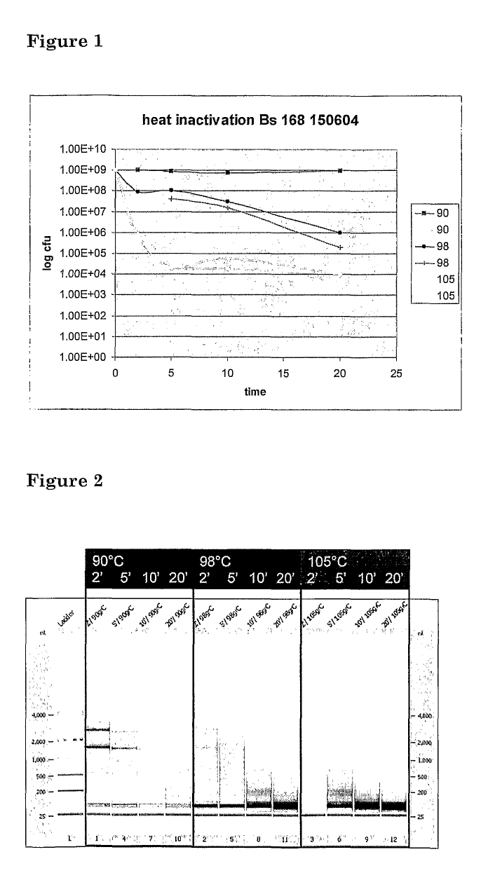

Results are shown in FIG. 1. It appears that spores of B. subtilis 168 were able to survive heat treatment of 90° C., but that survival declines at temperatures of 98° C. and higher.

### Example 2

### Degradation of rRNA

From the heat treated spores of Example 1 the RNA was isolated with a BIO101-FASTRNA Pro Blue kit (QBiogene #6025-050) according to the manufacturer's instructions, with the modification that the spores were processed 3 times 40 seconds in the FastPrep apparatus (QBiogene #6001-220) on setting 6 with 2 minutes of cooling on ice in between the lysis steps. After precipitation the RNA was treated with the TURBO DNA-free kit (AMBION #1907) according to the manufacturer's instructions. The integrity of the RNA was analysed on a RNA 6000 Nano LabChip (Agilent Technologies #5065-4476) with a 2100 bioanalyzer (Agilent Technologies #G2940CA).

At relatively low temperature treatments two clear bands are visible on the Bioanalyzer pseudogel, which indicate the presence of 16S and 23S rRNA, in a about 1:1.8 ratio (FIG. 2). The intensity of the bands becomes weaker at higher temperatures and/or longer treatment times. This coincides with the appearance of RNA material of low molecular weight.

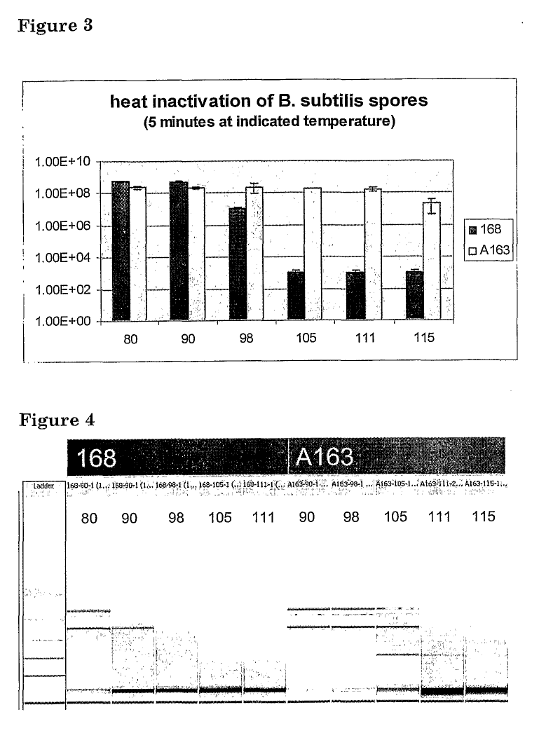

To investigate whether the degradation of RNA in the spores is dependent on the heat resistant properties of the spores, also the spores of a micro-organisms with a known higher heat resistance were tested. For this spores of the isolate B. subtilis A163 (Kort R, O'Brien AC, van Stokkum I H, Oomes S J, Crielaard W, Hellingwerf K J, Brul S. Assessment of heat resistance of bacterial spores from food product isolates by fluorescence monitoring of dipicolinic acid release. Appl Environ Microbiol. 2005 July; 71(7):3556-64). were chosen. Spores were generated and heat treated in the same way as the B. subtilis 168 spores of Example 1. The results are shown in FIGS. 3 and 4. While the viability of B. subtilis 168 spores decreases by 1 log after treatment at 98° C. for 5 minutes, there is no indication of loss of viability in B. subtilis A163 spores after this treatment. A comparable loss of viability in the latter spores only has been demonstrated at a temperature of 115° C. The integrity of the rRNA from B. subtilis 168 remained intact at 80° C., but degradation was observed at temperatures of 90° C. and higher (in concordance with the previous experiment). In contrast, the integrity of the RNA isolated from B. subtilis A163 appeared intact after treatments up to 98° C. With the 105° C. treatment degradation of the RNA was observed, predominantly of the 23S rRNA. Increasing degradation was observed at higher temperatures.

### Example 3

### Detection of mRNA in B. subtilis Spores

Using microarray analysis germination of B. subtilis spores has been studied. Hereto, a B. subtilis oligonucleotide library (Sigma-Genosys #BACLIB96) was spotted onto Corning GAPSII slides (Corning, #40003) according to standard protocols. Cy-labeled cDNA of spore RNA was made by direct incorporation of cy-labeled dUTP according to standard protocols. Hybridisation and washing of the micro-arrays was performed on an automated slide processor (Agilent) and scanning of the micro-array slides was done in an automated slide scanner (Agilent) according to the manufacturer's instructions.

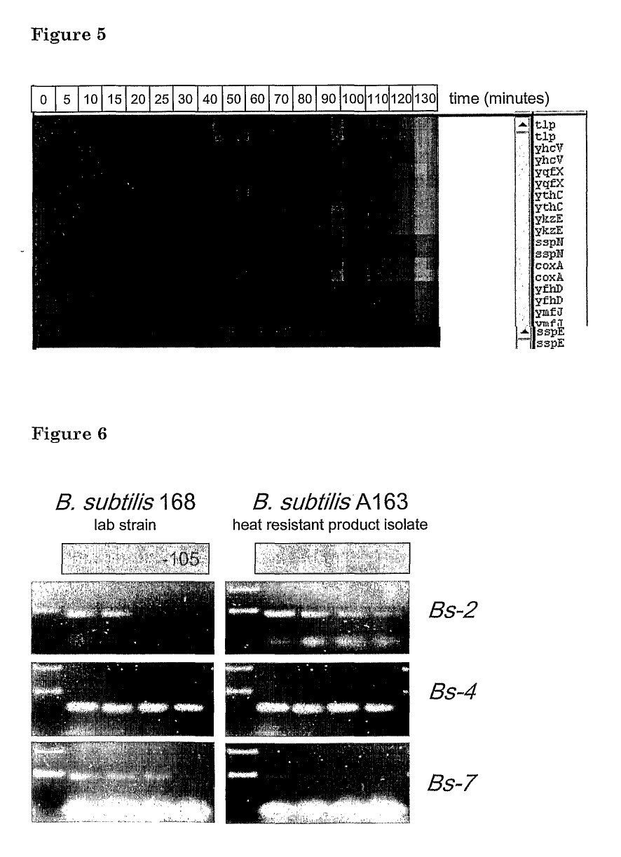

This showed (see FIG. 5) that a small group of mRNA molecules was present (about 20), of which the majority rapidly disappears during germination. Only sspE appeared to be present during the whole germination period. According to the fluorescence intensities of the hybridisation signals of the spore mRNA transcript levels were relatively high.

### Example 4

### Heat Degradation of mRNA

a. RT-PCR

To determine the stability of mRNA after heat treatment the spores were generated and treated as described in Example 1. RNA isolation was performed as described in Example 2. The isolated RNA was subjected to reverse transcription PCR(RT PCR) using Ready-to-Go RT-PCR Beads (Amersham Biosciences #27-9266-01) according to the manufacturer's instructions. Template concentration was between 10 and 200 ng. The primers used (determining 9 of the 20 transcription products) are indicated in Table 2.

TABLE 2
RT-PCR Primer sequences

| | | |
|---|---|---|
| Gene | Primer forward | Primer reverse |

| | | |
|---|---|---|
| TLP | TATCAGCAGCCTAATCCTG | CGTTTTGTCTCGCTGCAG |
| YHCV | GAGTTCAGTTAAAGATAC | ATACGAGTTCTGTTGACATC |
| YFHD | GGGCAGAAATCATATCC | CGCTCTGTTGTCGGCTG |
| YKZE | AACCGTCATAGCAGAGAC | TCAGGCTTGGTGACTTcc |
| SSPN | ATGGGAAACAACAAGAAAAAC | TCGCCTTTTGTCTGCATG |
| SSPE | GCTAACTCAAATAACTTCAGC | CAGCAGATTGGTTTTGCTG |
| COXA | GATACGCGCAATAACGGC | ATATGTTCCGTCAGTTGCC |
| YQFX | GAAGGTGGCAAACGATTAC | TGATGGACAAGGCTAAAGC |

TABLE 3

qPCR Primer and probe sequences. The dashes
indicate superbases added for stability of
the probe/primer, see http://www1.qiagen.
com/Products/Pcr/QuantiTect/CustomAssays.
aspx for details

| Gene | Forward | Reverse | FAM-labeled |
|---|---|---|---|
| Name | Primer | Primer | Probe |
| ykzE | GCAGAGACA | CAT*T*GT* | AGGTGCTGGAG |
| | TGCAAAATCA | AAT*CCCCG | GAAGAA |
| | TAA | AGTT | |
| coxA | ATAGACAGG | TCGTCAGCA | TAACCGAAACA |
| | GAGACGGAA | GTAACAC | CCACGA |
| | | GGT | |
| 16s | GGTCATTGG | CTACGCATT | GAAGAGGAGAG |
| rRNA | AAACTGGGA | T*CACCG | TGGAA |
| | | CTA | |
| 23s | CAGGTAACA | TTT*CGGAG | GATGAGGTGT |
| rRNA | CTGAATGGA | AGAACGAGC | GG*GTAG |
| | | TAT | |

Products were analysed with agarose gel electrophoresis using staining with ethidium bromide (FIG. 6). In untreated B. subtilis 168 spores for all transcription products, except for Tlp, amplification products could be detected. In untreated B. subtilis A163 spores a clear amplification product was obtained with primers for coxA, ykzE, yhcV, yfhD, and yqfX. A weak signal was obtained with sspE primers and no product was formed using Tlp and sspN primers. Possibly these transcripts do not occur in spores of this B. subtilis strain, or the primer sequences, which have been designed for B. subtilis 168 sequences, would not be suited for the A163 isolate.

In many cases in heat treated spores, a decrease in the amount of product was observed with increasing treatment temperatures (FIG. 6), which indicates degradation of mRNA. In spores of the A163 strain a similar decrease was observed, but starting at higher temperatures. For Bs-2 (the coxA transcript) no transcripts were detected in 168 spores at treatments temperatures of 98° C. and higher, while for A163 spores a decrease was only observed at temperatures of 115° C. Bs-4 (ykzE) did not show a clear decrease in the amount of product, which may be an indication that this transcript is less sensitive to heat treatment. Alternatively, the effect could be caused by the quantitative character of the RT-PCR experiment. To investigate this further for a limited set of RNAs a quantitative PCR (qPCR) was performed.

b. qPCR

B. subtilis 168 spores were heat treated at 98° C., the survival was measured by plating and culturing, and the spore RNA was isolated as described above. Reverse transcription of the spore RNA was performed using a RETROscript kit (Ambion, #1710) according to the manufacturer's instructions. Quantitative PCR was performed using a QuantiTect Multiplex PCR kit (Qiagen, #204543) on a 7500 Real-Time PCR System (Applied Biosystems). Primers and probes (see Table 2) were designed using the QuantiProbe Design Software (http://customassays.qiagen.com/design/inputsequences.asp). Two primer-probe sets were used to study the effects on the rRNA (16S and 23S). Two primer-probe sets were directed against the ykzE and coxA spore transcripts. In this type of experiments, the number of amplification cycles (Cₜ) required to reach a certain threshold level, is used to calculate the original amount of template. A titration curve with genomic DNA was used to calibrate. A ten times reduction in template concentration appeared to increase the number of cycles necessary to reach the threshold by three. This calibration was used to interpret the differences in Ct values between the treated and untreated spores.

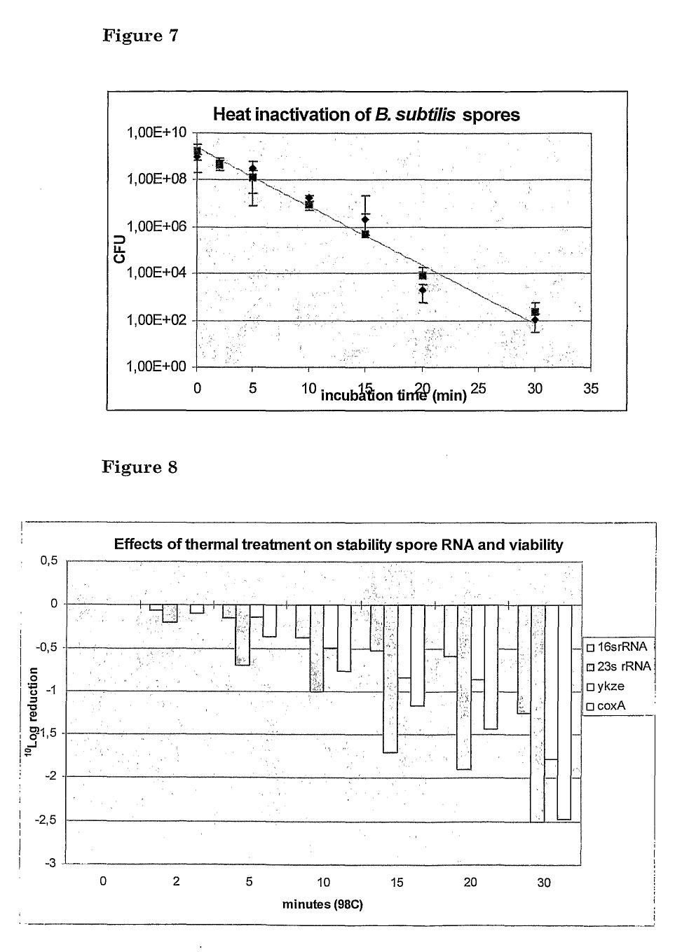

FIG. 7 shows the survival of heat treated spores according to the classical plating and culturing assay. FIG. 8 shows the results of the qPCR. It is clear that heat treatment results in a reduction of both the rRNAs and the mRNAs (which is in concordance with the earlier results on basis of gel electrophoresis). It also appears from this more sensitive qPCR that ykzE, which was deemed to be unresponsive in the RT-PCR assay, indeed degrades as a result of the heat treatment.

### Example 5

### Heat Inactivation of Clostridium botulinum Spores

Spores of C. botulinum strains NCTC 2916, 7272, 7273, 3806 and 10381 [Health Protection Agency: National Collection of Type Cultures Centre for Emergency Preparedness and Response: http://www.hpa.org.uk/srmd/div_cdmssd_nctc/default.htm] were generated by anaerobic cultivation in complex medium containing bacto peptone (50 g/ltr), trypticase peptone (5 g/ltr), K₂HPO₄ (1.25 g/ltr), NaHCO₃ (0.75 g/ltr), pH 7.2. The thus obtained spores were purified with 10 steps of washing with demineralised water at 4° C. The purity of the spores was monitored with a phasecontrast microscope (>99% phase bright). Heat treatment of the spores was done according to Kooiman et al. (“The screw cap tube technique: A new and accurate technique for the determination of the wet heat resistance of bacterial spores”. In: Spore Research, ed: Barker, A. N., Gould G. W. and Wolf, J., 1973, Academic Press, London, pp. 87-92). Survival was counted by plating serial dilutions of the samples in a peptone saline solution on Schaedler Anaerobic Agar (tritium microbiologie, the Netherlands http://www.tritium-microbiologie.nl/eindex.htm) Spores (10⁷ spores/ml) were heat treated by heating them for 5, 10 or 15 minutes at a temperature of 80° C., 105° C. or 110° C.

Survival of the spores at these temperatures is indicated in Table 1.

TABLE 1
Survival of C. botulinum spores at different heat treatments

| Treatment | 5 minutes | 10 minutes | 15 minutes |
|---|---|---|---|
| untreated | 10⁷ | | |
| 80° C. | | 10⁷ | |
| 105° C. | 58 | 0 | 0 |
| 110° C. | 0 | 0 | 0 |

### Example 6

### Degradation of rRNA

From the heat treated spores of Example 5 the RNA was isolated with a BIO101-FASTRNA Pro Blue kit (QBiogene #6025-050) according to the manufacturer's instructions, with the modification that the spores were processed 3 times 40 seconds in the FastPrep apparatus (QBiogene #6001-220) on setting 6 with 2 minutes of cooling on ice in between the lysis steps. After precipitation the RNA was treated with the TURBO DNA-free kit (AMBION #1907) according to the manufacturer's instructions. The integrity of the RNA was analysed on a RNA 6000 Nano LabChip (Agilent Technologies #5065-4476) with a 2100 bioanalyzer (Agilent Technologies #G2940CA).

At relatively low temperature treatments clear bands are visible on the Bioanalyzer pseudogel, The intensity of the bands becomes weaker at higher temperatures and/or longer treatment times. This coincides with the appearance of RNA material of low molecular weight.

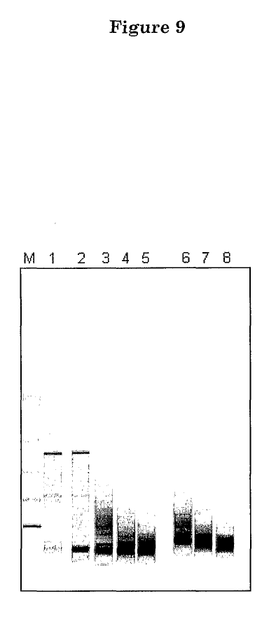

Results are shown in FIG. 9. It appears that spores of C. botulinum were able to survive heat treatment of 80° C., but that survival declines at temperatures of 105° C. and higher.

### Example 7

### Degradation of RNA Isolated from Heat Treated B. licheniformis ATCC 14580 Spores

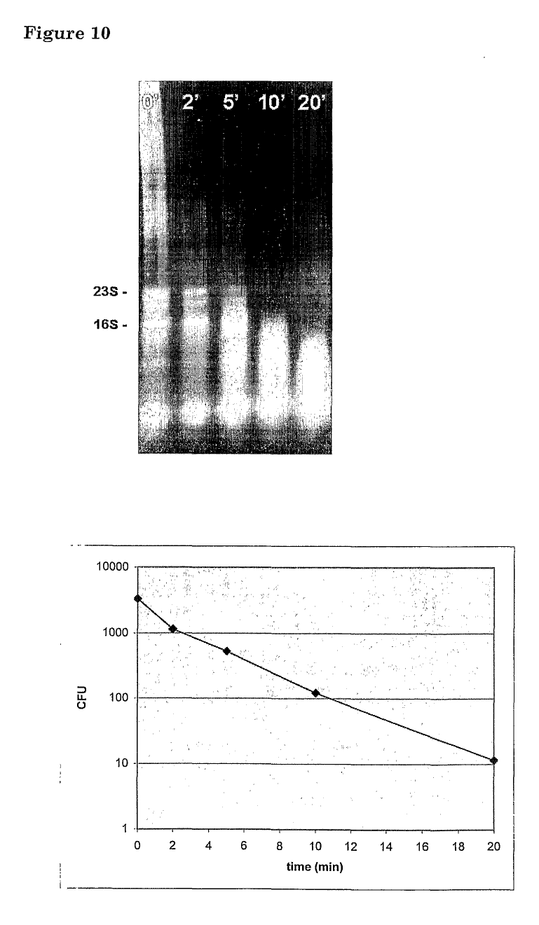

B. licheniformis ATCC 14580 (www.atcc.org) spores were treated for 2, 5 10 and 20 minutes at 90° C. Plate counts were used to establish the inactivation rate. Briefly, dilution series of spore suspensions were prepared in 0.1% peptone-0.85% NaCl and added to Trypticase soy agar pour plates. The number of colonies was counted after 4 days of incubation at 37° C. All heat inactivation experiments and viability counts were carried out in duplicate. RNa was isolates an the integrity was examined by denaturing agarose gel electrophoresis (Sambrook J, Fritsch E F, Maniatis T. 1989 Molecular Cloning, A Laboratory Manual, Second Edition. Cold Spring Harbour Laboratory Press). Spore viability decreased with the treatment time resulting in a decrease in viability counts. In parallel, RNA integrity was found to decrease during the thermal treatment of the spores. (see FIG. 10).

### Example 8

### Effects of UHP Treatment on Integrity of B. subtilis Spore RNA

B. subtilis 168 spores were treated for 2, 10 and 30 minutes at 600 MPa at an initial temperature of 50° C. Upon pressurization, the temperature adiabatically increased to approximately 6° C. Plate counts were used to establish the inactivation rate. Briefly, dilution series of spore suspensions were prepared in 0.1% peptone-0.85% NaCl and added to Trypticase soy agar pour plates. The number of colonies was counted after 4 days of incubation at 37° C. All heat inactivation experiments and viability counts were carried out in duplicate. RNA was isolated and the integrity was examined by Bioanalyzer (Agilent).

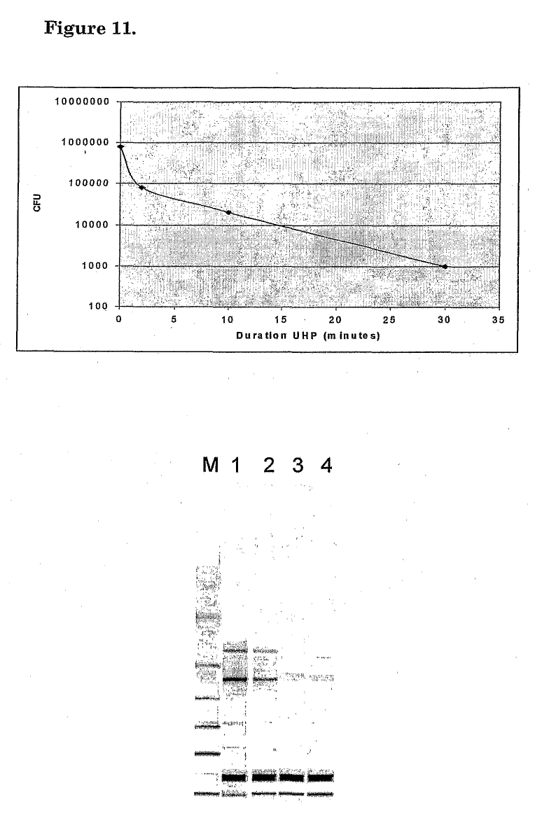

For results, see FIG. 11. Spore viability decreased with the treatment time resulting in a decrease in viability counts. In parallel, RNA integrity was found to decrease during the thermal treatment of the spores. The thermal treatment without an increase in pressure did not result in spore inactivation or loss of integrity of spore RNA.

1. Method to measure viability of preservation such as heat pressure, radiation, chemical or electrical treated bacterial spores comprising measuring the degree of degradation of RNA by said preservation treatment in said spores.

2. Method according to claim 1, wherein RNA is either ribosomal RNA (rRNA), messenger RNA (mRNA) or both.

3. Method according to claim 1, wherein the treatment is heat treatment.

4. Method to assay the effect of heat preservation methods by performing a method according to claim 1.

5. Method to assay bacterial contamination after heat, pressure, radiation, chemical or pulse electric preservation by performing a method according to claim 1.

6. Method according to claim 1, wherein the bacterial spores are from Bacillus and/or Clostridium species.

7. Heat preservation method comprising a) maintaining a sample at a certain temperature for a certain time; b) performing a method according to claim 1.

8. Use of RNA in microbial spores as a biomarker for viability.
